(19) United States
(12) Patent Application Publication
(10) Pub. No.: US 2020/0086115 A1
(43) Pub. Date: Mar. 19, 2020

(54) METHOD AND APPARATUS TO PERFORM STRUCTURAL AND MECHANICAL CHANGE TO JOINT CAPSULE, TENDON, AND LIGAMENT

(71) Applicant: THE REGENTS OF THE UNIVERSITY OF CALIFORNIA, Oakland, CA (US)

(72) Inventors: Brian Jet-Fei Wong, Irvine, CA (US); Dmitry Protsenko, Vallejo, CA (US); Tony Nguyen, Irvine, CA (US); Ranjan Gupta, Irvine, CA (US)

(73) Assignee: THE REGENTS OF THE UNIVERSITY OF CALIFORNIA, Oakland, CA (US)

(21) Appl. No.: 16/568,440

(22) Filed: Sep. 12, 2019

Related U.S. Application Data
(60) Provisional application No. 62/732,550, filed on Sep. 17, 2018.

(51) Int. Cl. A61N 1/20; A61N 1/36; A61N 1/04

(52) U.S. Cl. CPC A61N 1/205; A61B 5/4514; A61N 1/0416; A61N 1/36017

(57) ABSTRACT

A method of treating connective tissue by applying a DC current to the tissue in the absence of intentionally heating the tissue.

### CROSS REFERENCE TO RELATED APPLICATIONS

This application claims the benefit of and priority to U.S. provisional application No. 62/732,550 filed Sep. 17, 2018 which is incorporated herein by reference in its entirety.

### GOVERNMENT RIGHTS

This invention was made with government support under contract DR090349 awarded by the Department of Defense and under contract 1R21DE019026-01A2 awarded by the National Institutes of Health. The government has certain rights in the invention.

### BACKGROUND OF THE INVENTION

The present invention generally relates to the treatment of connective tissue. More specifically, the present invention relates to methods and apparatus to change the mechanical behavior and properties of connective tissue, such as capsular tissue, tendons, and ligaments.

Tendons, ligaments, and capsular tissues are important for providing stability and motion to the joints within the extremities. Current surgical treatment options generally rely upon open surgical approaches or arthroscopic techniques to gain access to these tissues. Many surgical procedures focus upon reducing the size, thickness, shape, or mechanical integrity of the tissue structure. For example, capsular tissue can be damaged and inflamed as joints age. At present, the only technology available that is commonly accepted to treat capsular tissue injury are conventional techniques such as incision or debridement.

Likewise, strengthening ligament or tendon in terms of enhancing its mechanical strength, (e.g., elastic modulus) is presently an elusive goal for orthopedic surgeons. At best, relatively primitive technologies involving clips or screws are used to alter the tension across these regions. In addition, surgeons currently use various sutures to change the dimensions of these tissue structures.

Beyond conventional surgical technologies, little has been developed in the medical technology to address the needs of surgeons who operate on the joints. Approximately two decades ago, thermal methods, such as radiofrequency, were advocated as a means of altering joint tissues including the capsule. However, the diffuse nature of these thermal interactions can bring about tissue injury, inflammation, and ultimately failure of the procedure. At present, there does not appear to be a company in the current market who still uses radiofrequency to address these issues related to ligament and capsular pathoanatomy.

As can be seen, there is a need in the marketplace for a technology that can lengthen or tighten joint tissue and simultaneously alter the mechanical behavior and properties of connective tissues. In instances where connective tissue must be treated, anesthesia and joint arthroscopy is often required. A minimally invasive technique that would rely upon surgical anatomy and needle-based technologies would be extremely effective in eliminating complicated procedures. Combining these solutions with an imaging technique such a CT, MRI, or ultrasound would be extremely compelling and valuable because procedures that can be performed in the office rather than the OR would be beneficial for reducing health costs and eliminating the need for invasive procedures.

### SUMMARY OF THE INVENTION

In one aspect of the present invention, a method of treating connective tissue, comprising applying a DC current to the tissue in the absence of heating the tissue.

In another aspect of the present invention, a method of treating connective tissue, comprising reducing hydrogen ions to hydrogen gas in a cathodic region of the tissue; and oxidizing hydroxide ions to oxygen gas in an anodic region of the tissue.

In a further aspect of the present invention, a method of strengthening connective tissue, comprising compressing or stretching the tissue; initiating, with the use of electrodes, electrochemical reactions in the tissue; and changing a water content in the tissue to alter mechanical properties of the tissue.

These and other features, aspects and advantages of the present invention will become better understood with reference to the following drawings, description and claims.

### BRIEF DESCRIPTION OF THE DRAWINGS

FIG. 1 is a schematic diagram of an experimental set up of elongation according to an embodiment of the present invention;

FIG. 2 are experimental test parameters of the embodiment of the present invention shown in FIG. 1;

FIGS. 3A-3B are graphs of load and displacement versus time according to embodiments of the present invention;

FIGS. 4A-4C are graphs of load and displacement versus time according to embodiments of the present invention;

FIGS. 5A-5B are graphs of Young's modulus and length according to embodiments of the present invention;

FIG. 6 is a schematic diagram of an experimental set up of compression according to a further embodiment of the present invention;

FIG. 7 are experimental test parameters of the embodiment of the present invention shown in FIG. 6;

FIGS. 8A-8C are graphs of Young's modulus, diameter, and thickness according to embodiments of the present invention;

FIGS. 9A-9B are photographs of diameter and thickness according to embodiments of the present invention;

FIG. 10 is a schematic diagram of an experimental set up of structural changes according to yet another embodiment of the present invention;

FIGS. 11A-11F are OCT images according to embodiments of the present invention;

FIG. 12 are depictions of a method according to an embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, the present invention provides a method of treating connective tissue such as capsular tissue, tendons, and ligaments. More particularly, the present invention generally employs a method of electromechanical reshaping (EMR). EMR uses an application of DC current to induce a shape change, while undergoing very little, unintended temperature changes, thus eliminating any tissue damage that may be caused by the diffuse nature of the heat source. Accordingly, no external heating source is applied to the tissue to intentionally cause a rise in tissue temperature.

Though the complete mechanism of EMR is not completely known, it is hypothesized that EMR works through electrochemical principles. At its simplest, EMR behaves similarly to an electrochemical cell. Because cartilage tissue is approximately 75% water, the predominant oxidation-reduction reactions are the reduction of hydrogen ions to hydrogen gas at the cathodic region and the oxidation of hydroxide ions to oxygen gas at the anodic region of the tissue. These hydrolysis reactions change the water content of the tissue, which can significantly alter mechanical properties in connective tissue. Furthermore, pH effects result from changes in the hydroxide and hydrogen ion concentration due to EMR redox effects.

### Examples

Elongation of Tendon

Methods

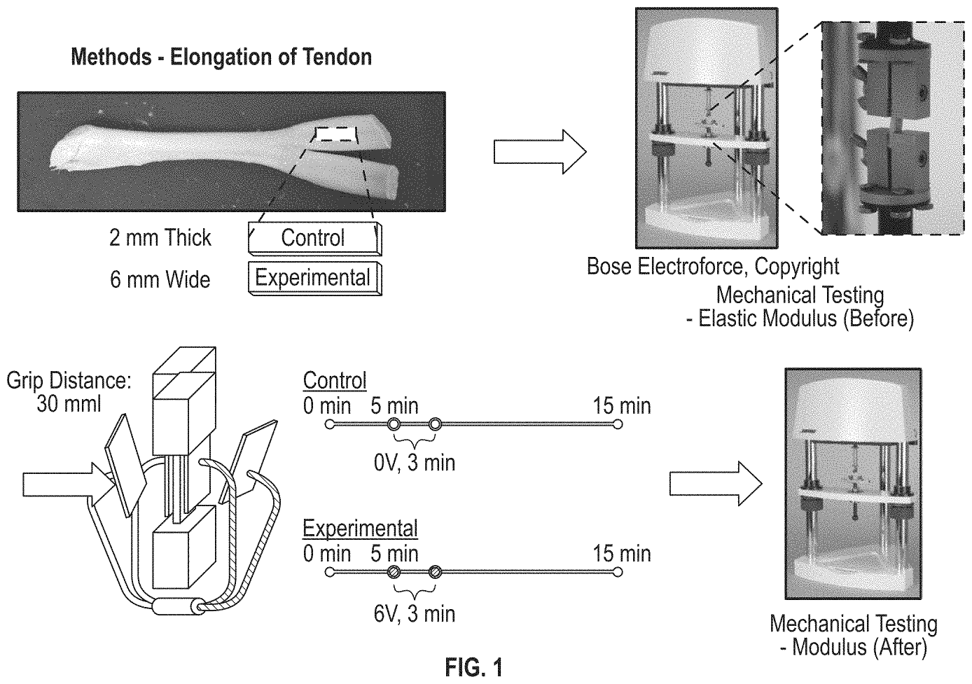

FIG. 1 schematically depicts an experimental setup.

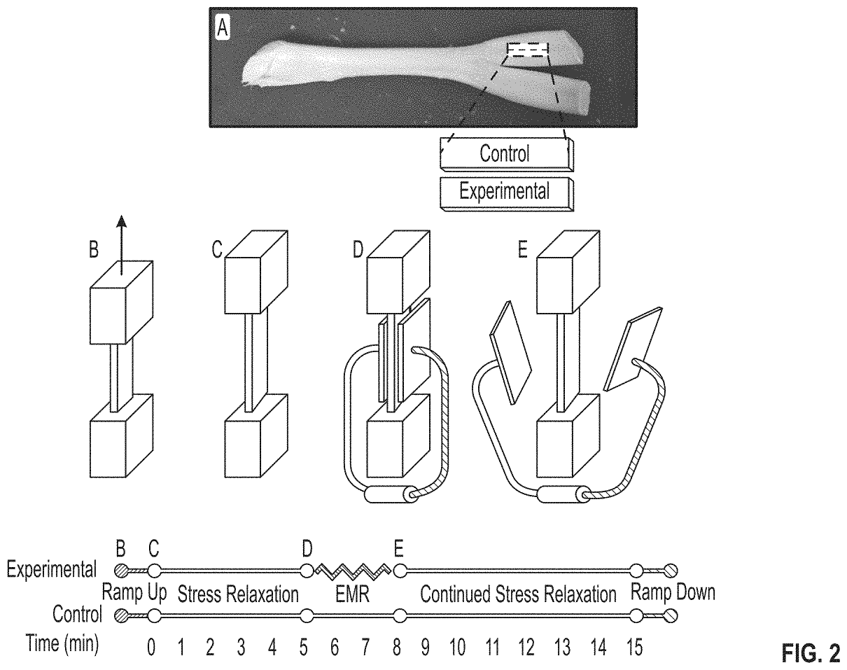

FIG. 2 further delineates the experiment in FIG. 1. Generally, in FIG. 2, a tendon specimen was elongated by the application of tension through the mechanical testing platform. This stretches the tendon. During this process, natural stress relaxation occurs as the displacement is now constant (C). Then, at (D), an electrical potential is applied using two flat metal electrodes (chiefly platinum). That is depicted by the wiggly line in the time scale at the bottom. During the entire time, stress is measured. Then, at (E), the electrodes are removed.

More specifically in FIG. 2, at (A), achilles tendon specimens were harvested from bovine leg specimens purchased from a local abattoir. Each tendon sample was cut into rectangular slices running the length of the tendon fiber at a thickness and width of 2±0.23 and 6±0.81, respectively. Adjacent samples were cut from the same specimen as a matched pair, with one designated a control that was exposed to the electrodes without the application of electric current, and one experimental sample. Another set of samples was cut to provide a control that was not in contact at all with the electrode plates. This served as a control for any external lateral compression of the tissue due to the electrode plates. Phosphate buffered saline solution (PBS) was used to hydrate the samples at pH 7.4 and to maintain the pH within a normal physiologic range.

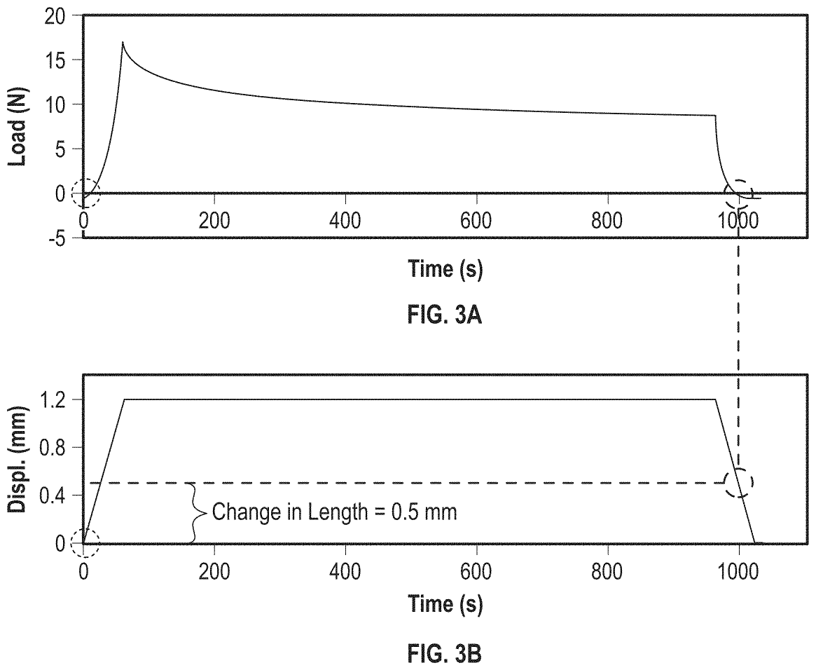

Referring back to FIG. 1, a specimen was placed on a mechanical testing apparatus (Electroforce 3200, Bose, Eden Prairie, Minn.) with clamps containing serrated edge grips such that the distance in between grips was 30 mm. The tissue was kept hydrated by periodic drips of PBS directly on the surface of the tissue during the time it was held in tension. Mechanical testing was done on the sample to assess changes in the Young's moduli before and after the tensile load and EMR was applied. The tissue was placed in a preconditioning step where a cyclic load was applied at approximately 2% strain and 1 Hz to remove crimps within the tissue fibers. The modulus was calculated from the data measured from the stress-strain curve of the last peak of the 20th cycle during the cyclic load. The ability for the tendon to stretch (changes in length) was determined by finding the differences in displacement between the clamps/grips at the time points of zero loads during the increase to 5% strain and the decrease to zero strain. Changes to the stress-strain curve and differences in stiffness and length were graphed. (FIGS. 3A-3B).

A tensile deformation was applied at a strain rate of 0.02 mm/s until the tissue length reached a strain of 5%. As the tissue was held at a constant strain for 15 minutes, the reaction force, displacement, and time was recorded. After reaching stress equilibrium at 5 minutes (after the tissue reached the 5% strain mark), the EMR process was initiated. Two flat platinum plates were placed on opposite sides of the sample and were held together by an insulated clamp to ensure that the entire surface of the electrode was in contact with the tissue. The leads of a DC power supply (E3646A, Agilent Technologies, Inc., Palo Alto, Calif.) were connected to the electrodes. Voltage amplitude and time duration were monitored through computer software (LabVIEW, National Instruments, Austin, Tex.). EMR was accomplished for 3 minutes at 6V. These parameters were chosen because they had previously demonstrated to produce significant mechanical behavior changes in similarly sized facial cartilage tissue in reshaping studies. The total time for the experiment was 15 minutes (including 3 minutes of active EMR). After this time interval, the tissue was returned to zero strain at a rate of 0.02 mm/s and immediately rehydrated in PBS for 15 minutes.

Mechanical testing was done again on the sample to determine the moduli after tensile load and EMR. The ability for the tendon to stretch (changes in length) was determined by finding the differences in displacement of the clamps/grips at the time points of zero loads during the increase to 5% strain and the decrease to zero strain. Changes to the stress-strain curve and differences in stiffness and length were graphed. Currently, mechanical testing using Material Testing Systems (MTS), the gold standard for orthopedic industry cases, has not been accomplished in this study, but will be utilized to further validate this technique.

Results

Noticeable changes in visual appearance during and after EMR were observed for experimental samples. During the duration of EMR, subtle changes to the tissue color and gas bubble formation were observed as noted in EMR of cartilage tissue. As compared to control samples and pre-EMR samples, the tissue became slightly translucent and on the side facing the cathode, the tissue surface was noticeably rougher in texture. After rehydration, the tendon samples retained its translucency.

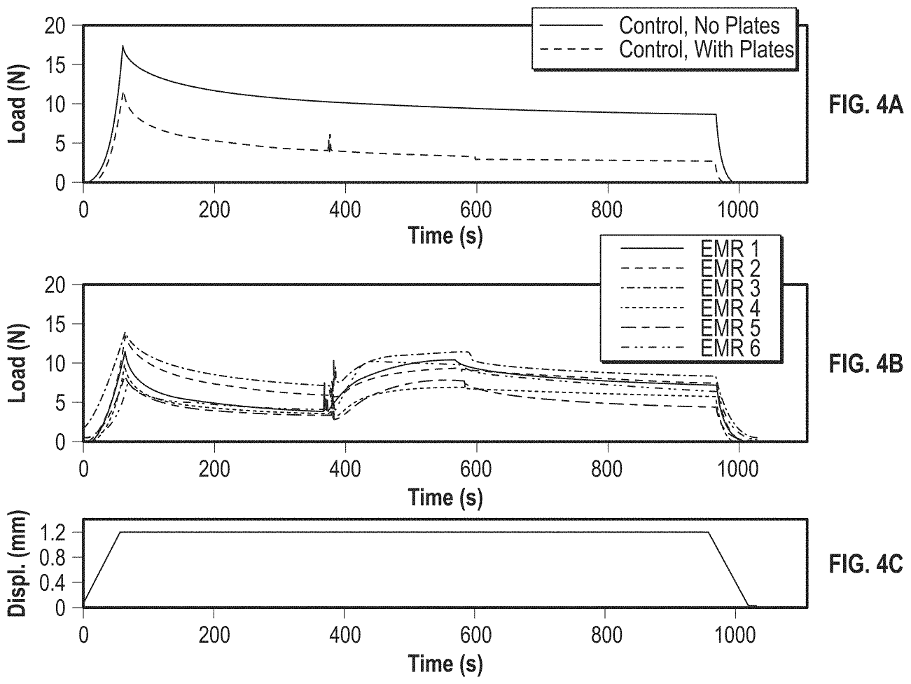

Mechanical testing during the elongation process of both control and EMR samples demonstrated stress relaxation of the tissue over the duration of load (FIGS. 4A-4C). FIG. 4A relates to controls without the application of the EMR effect (Voltage=zero). FIG. 4B shows what happens when electricity is applied and the EMR effect occurs. Notably the load changes and a different steady-state load is achieved.

When EMR was applied at 5 minutes, the reaction force increased due to the presence of gas bubbles pushing against the load cell, as shown by six EMR plots in the middle graph (FIG. 4B). This data agrees with the supposition of an oxidation-reduction process as a mechanism for EMR. In the top graph (FIG. 4A), two controls are shown in order to demonstrate that the physical contact of the electrode plates had little to no visible effect on the outcome of the mechanical testing. One designated control set was not contacted with electrode plates at all (Control, no plates), and another control set was exposed to electrodes without the application of electric current.

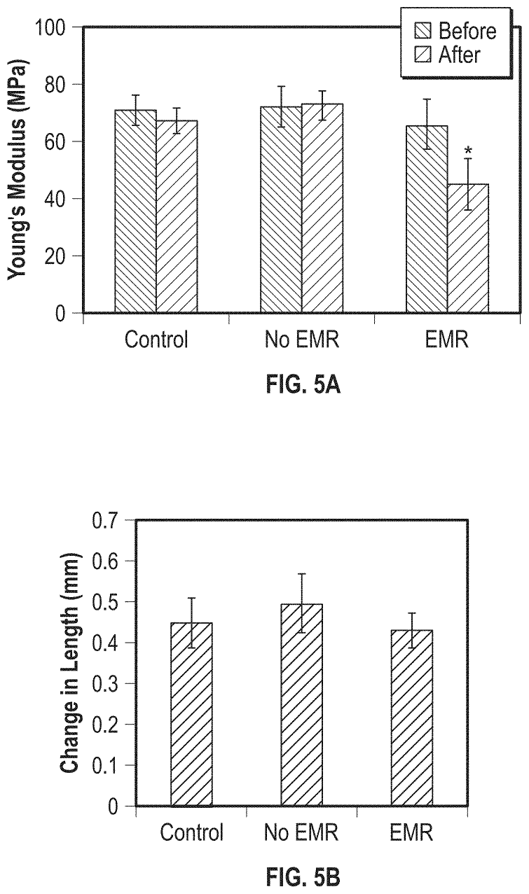

FIG. 5A shows Young's modulus for control and experimental samples pre-EMR and post-EMR plotted as average±standard error. N values are 10, 9, and 11 for control, no EMR, and EMR respectively. Error bars show standard error. Significance at p<0.05 is shown by the asterisk.

FIG. 5B shows change in length for control and experimental samples as a result of EMR plotted as average±standard error. No statistical significance (p<0.05) was shown between any of the controls and the EMR samples.

Post-EMR samples (FIG. 5A) were significantly softer than pre-EMR samples: 45.12±9.02 and 66.02±8.68, respectively (mean±standard error). Analysis using a two-sample t-test show a statistically significant softening of voltage-treated tendon (p=0.0049). There were no significant changes to the Young's modulus of either the control sample without electrode plate contact or the control sample without EMR applied (p=0.56 and p=0.99, respectively).

In addition, EMR does not affect the length of the tendon after elongation (FIG. 5B). A two sample t-test shows no statistically significant changes in length between the EMR sample and the control or the EMR sample and the “No EMR” control (p=0.52 and p=0.44, respectively). No significant difference between the two controls (p=0.89 for modulus and p=0.78 for length) demonstrate that the lateral compression due to the exposure of the tissue to the electrode plates has no effect on the modulus or the change in length.

Compression of Tendon

Methods

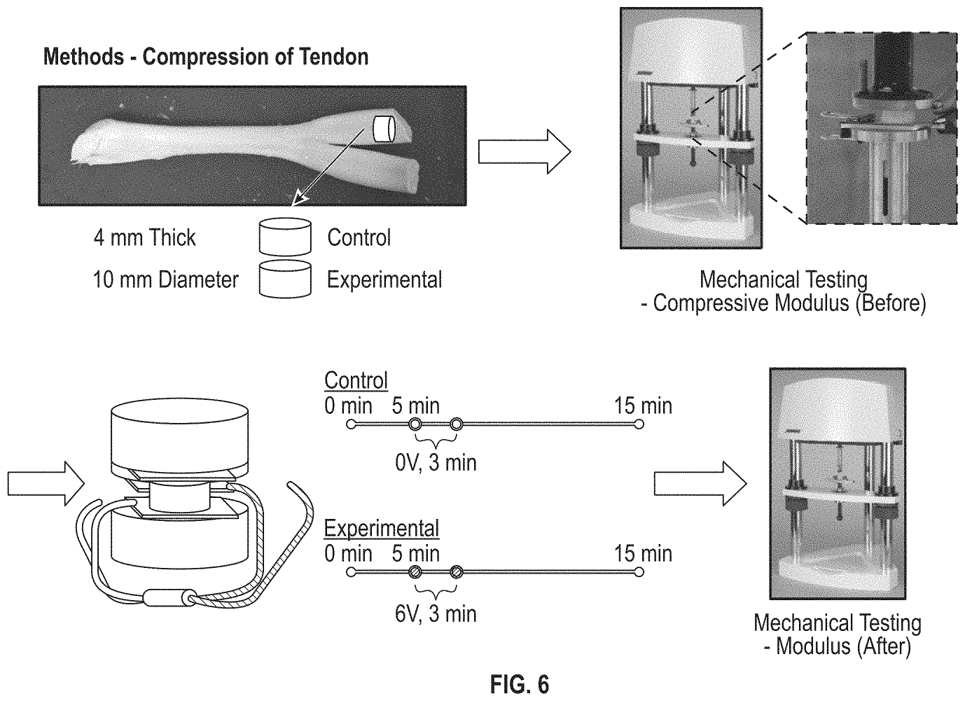

FIG. 6 schematically depicts an experimental setup. Generally, in FIG. 6, a sample of tendon is removed and compressed between two platens that are part of the mechanical testing platform. The photographs on the right are images of the mechanical testing device, and a close up of the testing interface (basically compressing the specimen). The application of electricity occurs for three minutes as shown on the time line in the center.

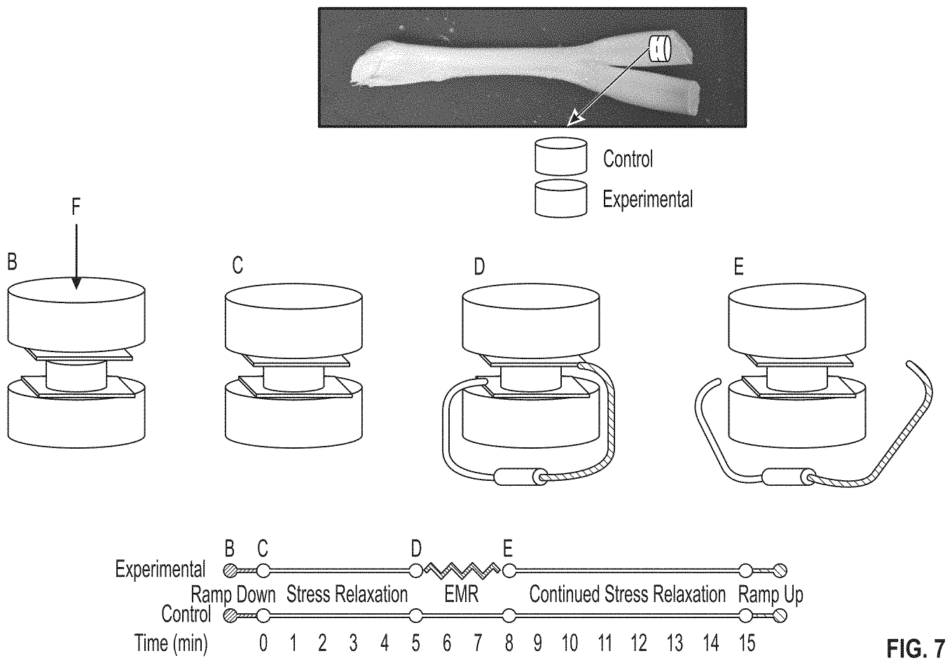

FIG. 7 further delineates the experiment in FIG. 6. Generally, in FIG. 7, a specimen is compressed at (B). Then, it is allowed to remain stable for a short period of time. During this time, natural stress relaxation occurs at (C). Then, a voltage is applied (D-E) which causes changes in tissue properties. Then, electrical current stops and the tissue continues to mechanically relax at (E).

More specifically, in FIG. 7, achilles tendon specimens were cut using a biopsy punch into cylindrical shapes with a height and diameter of 10±1.2 and 4±0.65 mm, respectively. Two pieces each were cut from the same specimen to serve as matched pairs for control and experimental samples. PBS was again used to hydrate and maintain the tissue at physiological pH.

The sample was placed on the mechanical testing apparatus fitted with compressive platens. The tissue was kept rehydrated by periodic drips of PBS during the duration of the applied load. A compressive deformation was applied at a rate of 0.02 mm/s until the tissue height reached a strain of 25%. As the tissue was held at a constant strain for 15 minutes, the reaction force, displacement, and time was continuously recorded.

After reaching stress equilibrium at 5 minutes after the tissue reached the 5% strain mark, EMR was applied for 3 minutes at 6 V. Platinum plates covered the entire top and bottom surface of the tissue during EMR process. The tissue remained under compression for another 7 minutes to allow further stress relaxation to complete. After the 15 minute procedure, the tissue was brought back to zero strain at a rate of 0.02 rnm/s and immediately rehydrated in PBS for 15 minutes outside of the platens.

Digital images (CanonRebel XSI DSLR) were acquired to observe changes in tissue shape after rehydration. Specifically, changes in the tissue height and diameter were assessed. Mechanical testing was done on the samples after rehydration to observe changes in the Young's moduli after the tensile load was applied. Changes to the stress-strain curve and differences in stiffness and length were graphed.

Results

Similar to elongation experiments, visible changes to the tendon were observed as a result of EMR applied under compression. Bubble formation and tissue translucency were noticed during EMR. The tissue retained its translucency after rehydration. Also, the surface exposed to the cathode electrode was noticeably rougher in texture.

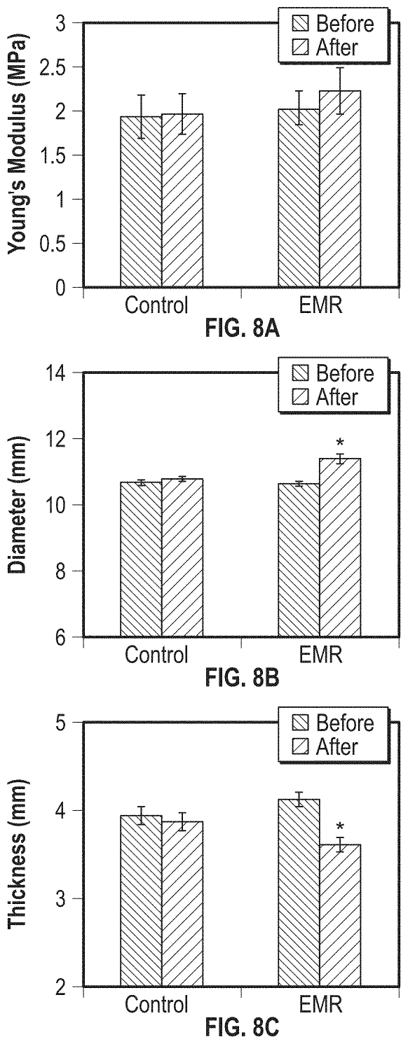

FIG. 8A shows Young's modulus for control samples before and after EMR during compression. Error bars show standard error, N=13. No significant difference can be observed between before and after EMR as a result of compression.

FIGS. 8B-8C show diameter and thickness of cylindrical samples for control and experimental before and after EMR. Compression experiment demonstrates a shortening effect caused by EMR as shown by a decrease in the thickness and an increase in the diameter. Error bars are standard error. Significance at p<0.05 is shown by the asterisks.

There were no significant differences in compressive modulus as a result of EMR (FIG. 8A). A two sample t-test showed no statistical significance between pre-EMR and post EMR samples for both control and experimental samples (p=0.81 and p=0.62, respectively).

Post-EMR samples showed a significant shape deformation in both the diameter and the thickness of the cylindrical shaped specimen (FIGS. 8B-8C). Post-EMR diameters were larger than pre-EMR diameters (FIG. 8B) indicating that the specimen flattened due to EMR (10.62±0.066 and 11.36±0.13, respectively). Post-EMR specimen thicknesses was reduced (FIG. 8C) indicating that EMR has a shortening effect on tendon (4.13±0.09 and 3.62±0.081, respectively). A two sample t-test showed statistically significant differences for both diameter and thickness as a result of EMR (p<0.05 and p<0.05, respectively), but no statistical difference for diameter and thickness of control samples (p=0.36 and p=0.47 respectively).

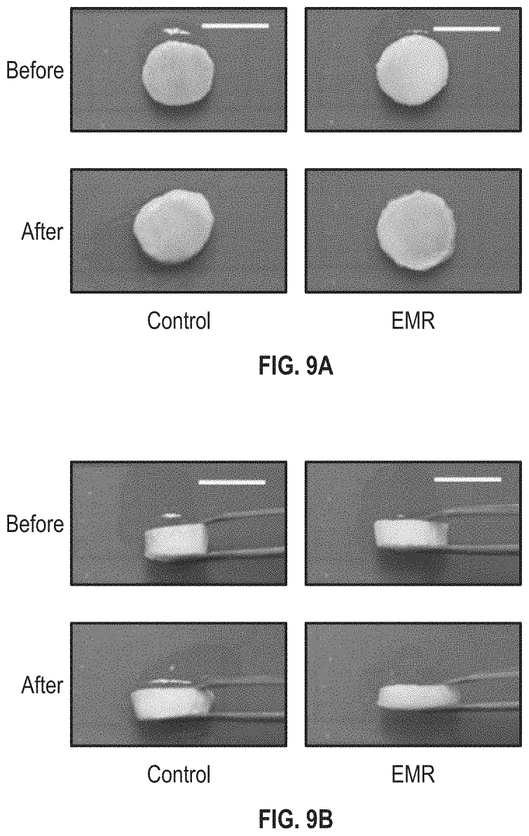

FIGS. 9A-9B are photographs of samples from FIG. 7. They show that diameter really does not change (FIG. 9A) and that thickness does change (FIG. 9B).

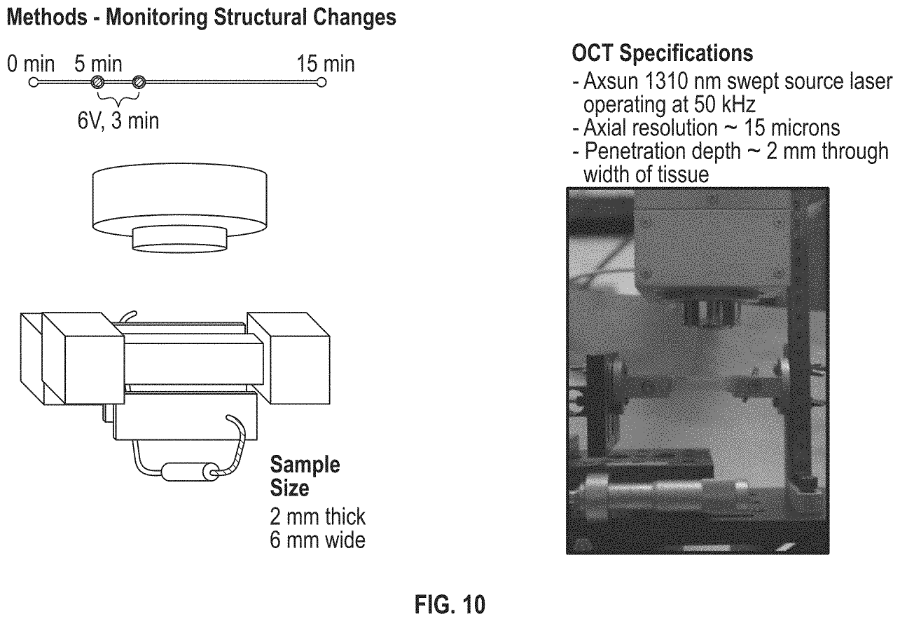

FIG. 10 is an experimental set up where structural changes are monitored using OCT imaging. The imaging device is placed above the specimen.

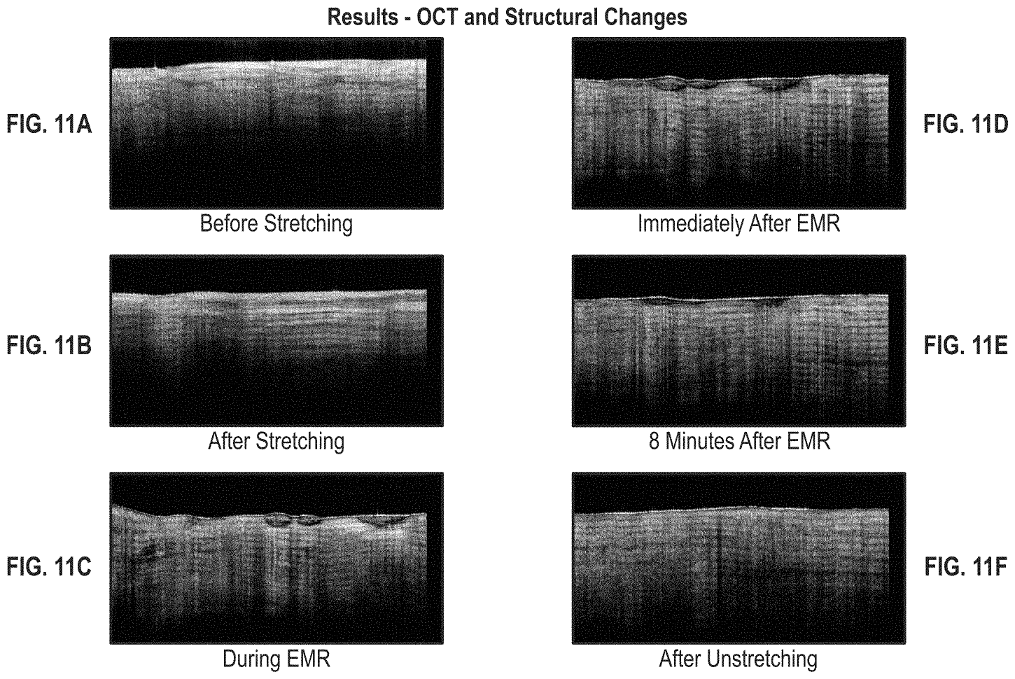

FIG. 11A-11F are OCT images from the FIG. 10 set up.

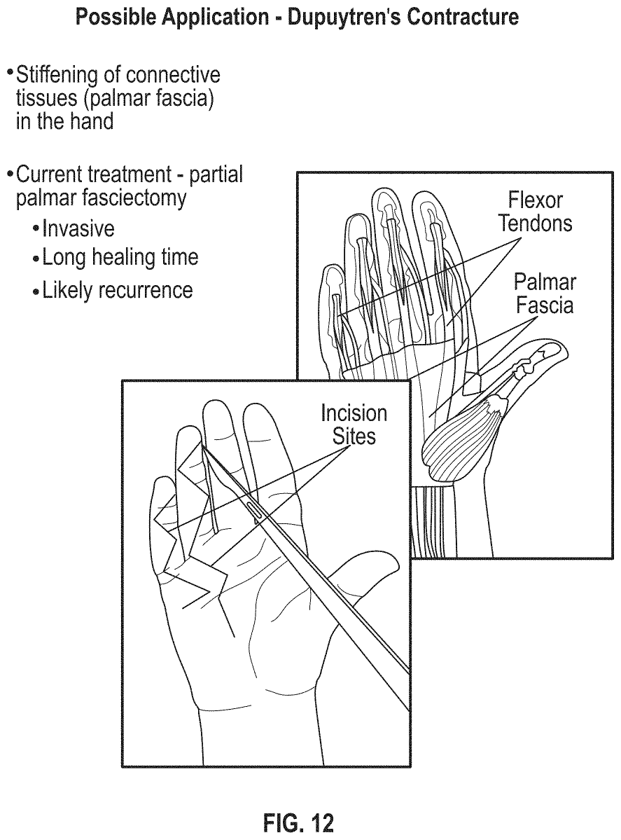

FIG. 12 graphically depicts a procedure according to an embodiment of the present invention. Instead of cutting contracted tendons (prior art), the present invention employs inserting an instrument through the skin and softening the tendon.

In sum, the present invention may provide the following advantages:
Minimally invasive
Less expensive
Easier to control and operate
Reduces the need for complicated and invasive procedures
Only requires the use of local anesthesia
Can be used with conventional surgical techniques
Relies upon low-cost disposables for revenue stream
Allows patient to have multiple incremental treatments overtime
Potentially widely used in developing world

It should be understood, of course, that the foregoing relates to exemplary embodiments of the invention and that modifications may be made without departing from the spirit and scope of the invention as set forth in the following claims.

We claim:

1. A method of treating connective tissue, comprising:
applying a DC current to the tissue in the absence of intentionally heating the tissue.

2. The method of claim 1, further comprising:
causing an oxidation-reduction reaction in the tissue.

3. The method of claim 1, further comprising:
reducing hydrogen ions to hydrogen gas in a cathodic region of the tissue.

4. The method of claim 1, further comprising:
oxidizing hydroxide ions to oxygen gas in an anodic region of the tissue.

5. The method of claim 1, further comprising:
compressing the tissue.

6. The method of claim 1, further comprising:
stretching the tissue.

7. A method of treating connective tissue, comprising:
reducing hydrogen ions to hydrogen gas in a cathodic region of the tissue; and
oxidizing hydroxide ions to oxygen gas in an anodic region of the tissue.

8. The method of claim 7, further comprising:
applying an electrical current to the tissue.

9. The method of claim 7, further comprising:
placing electrodes adjacent to the tissue.

10. The method of claim 7, further comprising:
placing an anode electrode on one side of the tissue; and
placing a cathode electrode on another side of the tissue.

11. The method of claim 7, further comprising:
avoiding the intentional heating of the tissue.

12. The method of claim 7, further comprising:
compressing the tissue.

13. The method of claim 7, further comprising:
stretching the tissue.

14. A method of strengthening connective tissue, comprising:
compressing or stretching the tissue;
initiating, with the use of electrodes, electrochemical reactions in the tissue;
changing a water content in the tissue to alter mechanical properties of the tissue.

15. The method of claim 14, further comprising:
reducing hydrogen ions to hydrogen gas in a cathodic region of the tissue.

16. The method of claim 14, further comprising:
oxidizing hydroxide ions to oxygen gas in an anodic region of the tissue.

17. The method of claim 14, further comprising:
sending a DC current through the tissue.

18. The method of claim 14, further comprising:
placing an anode electrode on one side of the tissue; and
placing a cathode electrode on another side of the tissue.

19. The method of claim 14, further comprising:
avoiding the intentional heating of the tissue.
